(19) United States
(12) Patent Application Publication
(10) Pub. No.: US 2023/0190697 A1
(43) Pub. Date: Jun. 22, 2023

(54) METHODS FOR TREATING VIRAL INFECTIONS WITH NAFAMOSTAT

(71) Applicant: ENSYSCE BIOSCIENCES, INC., La Jolla, CA (US)

(72) Inventor: Lynn KIRKPATRICK, La Jolla, CA (US)

(73) Assignee: ENSYSCE BIOSCIENCES, INC., La Jolla, CA (US)

(21) Appl. No.: 17/995,020

(22) PCT Filed: Mar. 31, 2021
(86) PCT No.: PCT/US2021/025146

§ 371 Date: Sep. 29, 2022

Related U.S. Application Data
(60) Provisional application No. 63/158,654, filed on Mar. 9, 2021.
(60) Provisional application No. 63/003,061, filed on Mar. 31, 2020.

(51) Int. Cl. A61K 31/245; A61P 31/14

(52) U.S. Cl. CPC A61K 31/245; A61P 31/14; A61K 9/0053

(57) ABSTRACT

Compositions and methods that can be used to treat or prevent a viral infection are described herein. For example, pharmaceutical compositions containing nafamostat mesylate may be used to treat infection by SARS-CoV-2 (“COVID-19”). Oral administration of nafamostat mesylate may be effective to reduce viral load in the gastrointestinal tract and reduce related GI symptoms.

### CROSS-REFERENCE TO RELATED APPLICATIONS

This application claims priority to and the benefit of U.S. Provisional Application No. 63/003,061, filed Mar. 31, 2020 and to U.S. Provisional Application No. 63/158,654 filed Mar. 9, 2021, both of which are incorporated herein in their entirety.

### BACKGROUND

Coronaviruses (CoV) are a group of highly diverse viruses that cause respiratory, enteric, hepatic and neurological diseases of varying severity and include severe acute respiratory syndrome coronavirus (SARS-CoV, Middle East respiratory syndrome coronavirus (MERS-CoV, MERS), and most recently severe acute respiratory syndrome coronavirus 2 (SARS-CoV-2). SARS-CoV-2 infection causes coronavirus disease 2019, or COVID-19.

The spectrum of clinical presentations of COVID-19 have ranged from asymptomatic infection to severe respiratory failure. Common complications of coronavirus infection, especially SARS-Cov-2 infection, include sepsis, respiratory failure, acute respiratory distress syndrome (ARDS), heart failure, septic shock, coagulopathy, acute cardiac injury, acute kidney injury, secondary infection, hypoproteinaemia, and acidosis. Respiratory failure from ARDS is the leading cause of mortality in COVID-19 patients.

There have been several populations of individuals that are now considered to be higher risk for serious complications of a SARS-CoV-2 infection. Those aged 65 or older, as well as those with cancer, chronic kidney disease, chronic obstructive pulmonary disease (COPD), down syndrome, heart failure, coronary artery disease, cardiomyopathy, sickle cell disease, type II diabetes mellitus, a BMI of 30 kg/m² or higher, an immunocompromised condition from organ transplant, are pregnant or who smoke have been identified as “high-risk” individuals based on the pathophysiology of the SARS-CoV-2 infection in these groups.

While the primary presentation in COVID-19 patients has been respiratory, some patients additionally present with digestive symptoms like diarrhea, nausea, vomiting, and abdominal pain (reported numbers range from 3.8% to 50.5%). Others present with only digestive symptoms. Indeed, chronic digestive conditions, such as irritable bowel syndrome (IBS) and inflammatory bowel disease (IBD) have also been identified as high-risk conditions that are associated with COVID-19 complications and less favorable outcomes during and after a SARS-CoV-2 infection. Digestive symptoms are not unique to the COVID-19 and usually present during infections with other respiratory syndrome viruses like SARS-CoV-1 and influenza.

Since the start of the COVID-19 pandemic, there have been many therapeutics introduced for the treatment of COVID-19 and its symptoms, however the vast majority of these require administration by a healthcare professional in a clinical (e.g., hospital) setting. For example, remdesivir has been approved by the FDA to treat COVID-19, but only in patients who have been hospitalized. The FDA has also approved several monoclonal antibody treatments, however, each require intravenous infusion, which must be done in a clinical setting.

The development of a treatment for a coronavirus infection that can be administered in an out-patient setting or a non-clinical setting (such as in one’s own home) would be an immense benefit, not only in the general treatment of the infection and disease from the perspective of the patient, but also for the healthcare system as a whole, since patients need not take up hospital space and resources to receive such a treatment.

### SUMMARY

The present disclosure provides a method of treating a viral infection in a subject comprising orally administering a therapeutically effective amount of nafamostat or a pharmaceutically acceptable salt thereof to the subject in need thereof, wherein the amount of nafamostat measurable in the plasma of the subject after 1 to 8 hours after administering is below about 5 ng/mL. The viral infection may be, for example, a coronavirus infection such as SARS-CoV, MERS-CoV, SARS-CoV-2, any mutation thereof, or any other coronavirus. A therapeutically effective amount of nafamostat may be, for example, 100 mg to about 1500 mg of nafamostat mesylate or an equivalent amount of nafamostat free base (i.e., about 45 mg to about 679 mg), or an equivalent amount of another pharmaceutically acceptable salt. The nafamostat may be administered, for example, in a solid oral dosage form, such as a tablet, a bi-layer tablet, a capsule, a multiparticulate, a drug-coated sphere, a matrix tablet, or a multicore tablet. A solid oral dosage for may comprise about 50 mg to about 200 mg of nafamostat mesylate or an equivalent amount of nafamostat or a pharmaceutically acceptable salt thereof.

The subject being treated for a viral infection may be, for example, a mammal such as a human. One example of a treatment regimen may include administering nafamostat in an amount equivalent to about 50 mg to about 500 mg nafamostat mesylate once, twice, or three times per day to the subject. Another example of a treatment regimen may include administering nafamostat in an amount equivalent to at least about 0.1 mg nafamostat mesylate per kg of the subject’s weight per day or in an amount equivalent to about 0.75 mg nafamostat mesylate per kg of the subject’s weight per day to about 20 mg per kg of the subject’s weight per day or in an amount equivalent to about 1 mg nafamostat mesylate per kg of the subject’s weight per day to about 10 mg nafamostat mesylate per kg of the subject’s weight per day.

Advantageously, the solid oral dosage form comprising nafamostat or a pharmaceutically acceptable salt hereof may be administered by the subject themselves, in a home or out-patient setting.

The present disclosure additionally provides a method of inhibiting a viral infection in a subject, the method comprising orally administering a therapeutically effective amount of nafamostat or a pharmaceutically acceptable salt thereof, wherein the amount of nafamostat measurable in the plasma of the subject after 1 to 8 hours after administering is below about 5 ng/mL. The administering, for example, may occur before a symptom of a viral infection is observed in the subject and serve in a preventative or prophylactic manner. A subject may be administered the nafamostat or pharmaceutically acceptable salt thereof, for example, after exposure or presumed exposure to a virus.

The viral infection may be, for example, a coronavirus infection such a, SARS-CoV, MERS-CoV, SARS-CoV-2, or another mutated coronavirus. A therapeutically effective amount of nafamostat may be, for example, an amount of nafamostat that equivalent to the amount of nafamostat in about 100 mg to about 1500 mg of nafamostat mesylate. The nafamostat may be administered, for example, in a solid oral dosage form, such as a tablet, a bi-layer tablet, a capsule, a multiparticulate, a drug-coated sphere, a matrix tablet, or a multicore tablet. A solid oral dosage for may comprise about 50 mg to about 200 mg of nafamostat mesylate or an equivalent amount of nafamostat or a pharmaceutically acceptable salt thereof.

The subject being treated for a viral infection may be, for example, a mammal such as a human. A subject may be aged 65 or older and/or have one or more of cancer, chronic kidney disease, chronic obstructive pulmonary disease (COPD), down syndrome, heart failure, coronary artery disease, cardiomyopathy, sickle cell disease, type II diabetes mellitus, a BMI of 30 kg/m2 or higher, an immunocompromised condition from organ transplant, pregnancy, a smoking habit, inflammatory bowel disease, and irritable bowel syndrome. A subject may have one or more of moderate-to-severe asthma, cerebrovascular disease, cystic fibrosis, hypertension, liver disease, dementia, Alzheimer’s Disease, pulmonary fibrosis, thalassemia, type I diabetes mellitus, a BMI of 25 kg/m² to 30 kg/m², and an immunocompromised condition from blood or bone marrow transplant, immune deficiency, HIV, or use of corticosteroid or other immune-weakening medicine.

One example of a treatment regimen may include administering nafamostat in an amount equivalent to about 50 mg to about 500 mg nafamostat mesylate once, twice, or three times per day to the subject. Another example of a treatment regimen may include administering nafamostat in an amount equivalent to at least about 0.1 mg nafamostat mesylate per kg of the subject’s weight per day or in an amount equivalent to about 0.75 mg nafamostat mesylate per kg of the subject’s weight per day to about 20 mg per kg of the subject’s weight per day or in an amount equivalent to about 1 mg nafamostat mesylate per kg of the subject’s weight per day to about 10 mg nafamostat mesylate per kg of the subject’s weight per day.

Advantageously, the solid oral dosage form comprising nafamostat or a pharmaceutically acceptable salt hereof may be administered by the subject themselves, in a home or out-patient setting.

The present disclosure additionally provides a method of reducing the risk of experiencing an adverse event associated with intravenous administration of nafamostat mesylate in a subject having a viral infection comprising orally administering a therapeutically effective amount of nafamostat mesylate to the subject, wherein the adverse event is one or more of allergic reaction, diabetic ketoacidosis, agranulocytosis, and hyperkalemia. The nafamostat may be administered, for example, in a solid oral dosage form, such as a tablet, a bi-layer tablet, a capsule, a multiparticulate, a drug-coated sphere, a matrix tablet, or a multicore tablet. A solid oral dosage for may comprise about 50 mg to about 200 mg of nafamostat mesylate or an equivalent amount of nafamostat or a pharmaceutically acceptable salt thereof.

### DETAILED DESCRIPTION

There is a growing body of scientific evidence which shows that the SARS-CoV-2 virus stays in the gut for an extended period of time, even after the virus is no longer detectable in respiratory samples. While the majority of COVID-19 patients primarily present with respiratory symptoms, there is a substantial subset that also report digestive symptoms. According to a recent study, 53% of patients hospitalized with COVID-19 reported digestive symptoms (Elmunzer, J.B., et al.; Digestive Manifestations in Patients Hospitalized with Coronavirus Disease 2019; Clinical Gastroenterology and Hepatology; Sep. 30, 2020). Around 20% of those with COVID-19 only report experiencing digestive symptoms. As more data is collected on infected subjects and their subsequent symptoms, the categorical nature of the symptoms are leading to speculation that there are two different subtypes of SARS-CoV-2 manifestations referred to as “gut-tropism” and “lung-tropism,” depending on where the virus enters the body (i.e. inhaled or ingested) and becomes established, and thus where symptoms develop.

Once inside the body, the virus must gain entry into a host cell in order to replicate. One way by which a coronavirus gains entry into a host cell is by binding to angiotensin converting enzyme 2 (ACE2) facilitated by an S-spike protein on the viral surface. Subsequent cleavage of the S-spike by a protease, such as transmembrane serine protease 2 (TMPRSS2) facilitates fusion of the viral envelope with the host cell. GI tissues, like the lungs, are lined with cells containing ACE2 receptors and therefore are ideal conditions for sustaining SARS-CoV-2 infection. In fact, there are about 100 times more ACE2 receptors in the GI tract than in the respiratory organs. ACE2 is overexpressed in the gut in inflammatory bowel conditions, such as IBS and IBD, which might explain the observed increased complications and unfavorable outcomes during and after coronaviral infection in individuals with these conditions.

It has been hypothesized that an enteric viral infection might modulate the severity of pulmonary and system illness through alterations in the microbiome, dysregulated intestinal immunity and/or increased gut permeability. The etiology of GI symptoms may be linked to the cytokine release that occurs when the virus leaves an infected cell. GI symptoms could also be caused by viral destruction of GI tissues.

As more data is collected on symptoms and subsequent outcomes, there have been several reports that that those experiencing GI symptoms are more likely to develop negative health complications or risks. For example, a study from November 2020 observed that subjects have a greater risk of developing acute respiratory distress syndrome (ARDS) and a higher risk of needing mechanical ventilation and tracheal intubation when reporting GI symptoms (Chen, R., et al.; Gastrointestinal Symptoms Associated with Unfavorable Prognosis of COVID-19 Patients: A Retrospective Study; Frontiers in Medicine; 11 Nov. 2020).

The GI tract, therefore, emerges as a potential target for treating coronaviral infections. Inhibition or reduction of viral replication in the GI tract may, in some patients, be the key to fighting the infection as well as preventing transmission to others through GI-related routes.

Disclosed herein is one such method which has promise in the treatment of coronaviral infections by targeted administration to the GI tract and inhibition of a critical mechanism by which the coronavirus enters a host cell. While not wishing to be bound by any theory, it is believed that orally-administered nafamostat (or a pharmaceutically acceptable salt thereof, such as nafamostat mesylate) is able to inhibit viral replication and infection by blocking binding of the virus to the ACE2 receptor on the host cell and by blocking the activity of TMPRSS2. Further, since this mechanism of inhibition targets receptors on the host cell and not the virus itself, nafamostat has the potential to be widely effective against any virus that utilizes one or both of ACE2 and TMPRSS2 to infiltrate the host cell, including other coronaviruses (e.g., SARS-CoV and MERS-CoV).

Additionally, nafamostat is thought to be particularly effective as it exhibits additional properties that may be particularly beneficial in treating various physiological abnormalities associated with coronavirus infections and their diseases. For example, nafamostat has antiinflammatory effects, which may help mitigate the cytokine storm syndrome associated with COVID-19. Nafamostat is also an anti-coagulant, which may reduce or treat cardiovascular complications. Nafamostat is also a mucolytic and may aid in the clearance of mucous and therefore the clearance of the virus.

The invention described herein is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The invention is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various methods are provided herein for the treatment of a viral infection in a subject with nafamostat. Nafamostat is a broad-spectrum synthetic serine protease inhibitor having a chemical structure as depicted below in Formula (I):

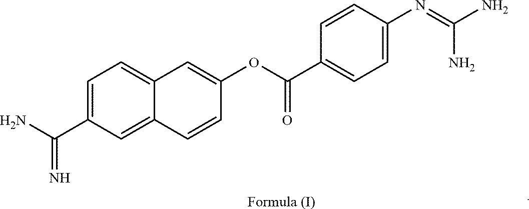

Methods of treating a viral infection with nafamostat may utilize nafamostat in any pharmaceutically acceptable form. For example, in any embodiment, the nafamostat may be nafamostat free base. In other embodiments, the nafamostat may be a pharmaceutically acceptable salt of nafamostat including, but not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 5 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion (e.g., an alkali metal ion, an alkaline earth ion, or an aluminum ion); or coordinates with an organic base such as ethanolamine, diethanolamine, triethanolamine, N-methylglucamine and the like. In any embodiment, the salt of nafamostat may be nafamostat mesylate.

Nafamostat mesylate, which is chemically known as 6-amidino-2-naphthyl p-guanidinobenzoate dimethanesulfonate, is shown below as Formula (Ia):

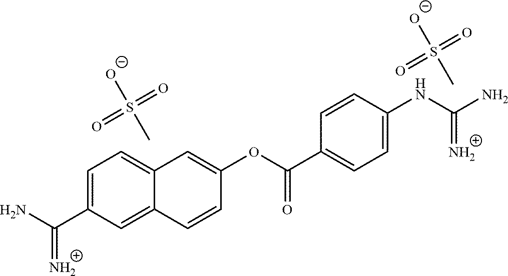

.

Nafamostat mesylate has been approved for intravenous, intra-arterial, or extracorporeal use in Japan and Korea. Nafamostat mesylate has been approved by the FDA for the treatment of various conditions. Therefore, therapeutically effective amounts of nafamostat for use in the methods disclosed herein will be described as therapeutically effective amounts of nafamostat mesylate, however one of skill in the art will be able to calculate an equivalent amount of nafamostat free base using the molecular weight of nafamostat mesylate (347.37 g/mol) and nafamostat free base (157.17 g/mol) to determine an equivalent amount of free base. Likewise, one of ordinary skill in the art would be able to calculate an equivalent amount in another salt form using its molecular weight. As used herein the term “therapeutically effective amount” is an amount of an active agent which confers a therapeutic effect on the treated subject, which may be prophylactic or retroactive to the onset of the condition being treated. The therapeutic effect may be objective (e.g., measurable by a quantitative or qualitative test or marker) or subjective (e.g., subject gives an indication of or feels an effect or physician observes a change).

Any embodiment reciting the inclusion or use of nafamostat mesylate also includes nafamostat free base or any other pharmaceutically acceptable salt. Likewise, any embodiment reciting inclusion or use of nafamostat also includes the nafamostat mesylate or any other pharmaceutically acceptable salt of nafamostat. Nafamostat (or nafamostat mesylate) may be used in any crystalline, semi-crystalline, or amorphic form. Nafamostat may also be used in a solvated or hydrated form. Nafamostat mesylate is commercially available from many sources, such as Haihang Industry Co. (Shandong, China), Katsura Chemical Company (Kanagawa, Japan), and Sigma-Aldrich (St. Louis, MO, USA).

There is emerging speculation that there may be two different subtypes of COVID-19 manifestations or “tropisms” depending on where the virus enters the body and therefore dictating the range of symptoms. The most commonly reported reflects “lung-tropism” or the inhalation of the virus into the lungs. However, another substantive route may be through ingestion of the virus, leading to a “gut tropism.”

In one embodiment, the present disclosure provides a method of treating a viral infection in a subject comprises orally administering a therapeutically effective amount of nafamostat or a pharmaceutically acceptable salt thereof (e.g., mesylate) to a subject in need thereof. In any embodiment, the viral infection may be a coronavirus infection, such as a SARS-CoV infection, a MERS-CoV infection, or a SARS-CoV-2 infection.

Advantageously and surprisingly, administration of nafamostat to the gastrointestinal (GI) tract via oral administration has favorable pharmacokinetic properties when compared with other administration routes, such as intravenous administration. In particular, the rapid metabolism of intravenously administered nafamostat in the blood and liver rapidly diminishes its potency after bolus administration (estimated t_{½} of 5 to 23 minutes). However, orally administered nafamostat is able to exert therapeutic efficacy against a viral infection, such as a coronavirus, without requiring therapeutically effective system circulation. Therapeutic efficacy may be observed where there little to no detectable nafamostat in the blood, such as a Cₘₐₓ below 5 ng/mL. Instead, nafamostat will achieve therapeutically effective levels within the gastrointestinal tract. This more direct oral route of administration bypasses the need to use the blood as a drug delivery conduit and represents a more efficient use of nafamostat, thereby reducing the amount of nafamostat needed to impart a therapeutic effect. Further, avoiding systemic introduction reduces the risk of off-target and adverse events.

A therapeutically effective amount of orally-administered nafamostat mesylate, as disclosed herein, may be specified on a per-administration basis, for example, about 50 mg to about 500 mg of nafamostat mesylate per administration, such as about 50 mg to about 400 mg, about 50 mg to about 300 mg, about 50 mg to about 200 mg, about 50 mg to about 100 mg, about 100 mg to about 500 mg, about 100 mg to about 400 mg, about 100 mg to about 300 mg, about 100 mg to about 200 mg, about 200 mg to about 500 mg, about 200 mg to about 400 mg, about 200 mg to about 300 mg, about 300 mg to about 500 mg, about 300 mg to about 400 mg, or about 400 mg to about 500 mg, as well as any value there between, such as about 75 mg, about 100 mg, about 125 mg, about 150 mg, about 175 mg, about 200 mg, about 225 mg, about 250 mg, about 275 mg, about 325 mg, about 350 mg, about 375 mg, about 400 mg, about 425 mg, about 450 mg, about 475 mg, or about 500 mg. A therapeutically effective amount may be administered by any effective dosing regimen, such as once a day, twice a day, three times a day, four times a day, five times a day, or at another interval.

Examples of intervals that may be used, for example, with a modified (e.g., controlled or extended) release dosage form include about 1 day or longer, about 2 days or longer, about 3 days or longer, about 4 days or longer, about 5 days or longer, about 6 days or longer, about 7 days or longer, or about 2 weeks or longer. In practicing methods of the present disclosure, treatment regimens may include two or more dosage intervals, such as three or more dosage intervals, such as four or more dosage intervals, such as five or more dosage intervals, including ten or more dosage intervals. The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals.

In another example, a therapeutically effective amount of nafamostat mesylate may be specified on a per-day basis, for example, about 150 mg to about 1500 mg of nafamostat mesylate per day, such as about 150 mg to about 1200 mg, about 150 mg to about 900 mg, about 150 mg to about 600 mg, about 150 mg to about 300 mg, about 300 mg to about 1500 mg, about 300 mg to about 1200 mg, about 300 mg to about 900 mg, about 300 mg to about 600 mg, about 600 mg to about 1500 mg, about 600 mg to about 1200 mg, about 600 mg to about 900 mg, about 900 mg to about 1500 mg, about 900 mg to about 1200 mg, or about 1200 mg to about 1500 mg, as well as any value there between, such as about 175 mg, about 200 mg, about 225 mg, about 250 mg, about 275 mg, about 300 mg, about 325 mg, about 350 mg, about 375 mg, about 425 mg, about 450 mg, about 475 mg, about 500 mg, about, 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg (1 g), about 1025 mg, about 1050 mg, about 1075 mg, about 1100 mg, about 1125 mg, about 1150 mg, about 1175 mg about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, or about 1500 mg. When administered in two or more daily doses, the amount in each administered dose can be added together to yield a total daily dose.

In any embodiment, dosing (amount and/or frequency) may be varied depending upon a variety of factors including subject species, age, body weight, general health, gender and diet of the subject, the mode and time of administration, rate of excretion, drug combination, and severity of the viral infection.

For example, a therapeutically effective amount of nafamostat mesylate may be administered to a subject in an amount based on the subject’s weight. Specific examples of therapeutically effective amounts of nafamostat mesylate, based on subject weight, include about 0.75 mg/kg/day to about 20 mg/kg/day, such as about 0.75 mg/kg/day to 7.5 mg/kg/day, 0.75 mg/kg/day to 5 mg/kg/day, about 0.75 mg/kg/day to about 2.5 mg/kg/day, about 1 mg/kg/day to about 15 mg/kg/day, about 1 mg/kg/day to about 10 mg/kg/day, about 1 mg/kg/day to about 7.5 mg/kg/day, about 1 mg/kg/day to about 5 mg/kg/day, about 1 mg/kg/day to about 2.5 mg/kg/day, about 2.5 mg/kg/day to about 15 mg/kg/day, about 2.5 mg/kg/day to about 10 mg/kg/day, about 2.5 mg/kg/day to about 7.5 mg/kg/day, about 2.5 mg/kg/day to about 5 mg/kg/day, about 5 mg/kg/day to about 15 mg/kg/day, about 5 mg/kg/day to about 10 mg/kg/day, about 5 mg/kg/day to about 7.5 mg/kg/day, about 7.5 mg/kg/day to about 10 mg/kg/day, or about 7.5 mg/kg/day to about 15 mg/kg/day. Specific examples include about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 11 mg/kg/day, about 12 mg/kg/day, about 13 mg/kg/day, about 14 mg/kg/day, about 15 mg/kg/day, about 16 mg/kg/day, about 17 mg/kg/day, about 18 mg/kg/day, about 19 mg/kg/day, and any range between any two of these values.

A therapeutically effective amount of nafamostat or a pharmaceutically acceptable salt thereof (e.g., mesylate) may be included in a solid dosage form for oral administration. Therefore, in another embodiment, the present disclosure provides a method for treating a viral infection in a subject in need thereof comprising orally administering a solid dosage form comprising nafamostat or a pharmaceutically acceptable salt thereof such as nafamostat mesylate. Advantageously, by providing nafamostat in a solid oral dosage form, a subject may self-administer the dosage form, as directed by a health provider, in an out-patient non-hospital setting, simplifying a treatment regimen over using nafamostat, for example, in a parenteral dosage form for intravenous administration. Notably, however, orally administered Nafamostat will exert therapeutic effects in treating a viral infection, such as coronavirus, without achieving a therapeutically effective level systemically (in the plasma), such as achieving a Cₘₐₓ below 5 ng/mL; rather, Nafamostat will achieve therapeutically effective levels within the gastrointestinal tract.

In any embodiment, a solid oral dosage form may be an immediate release dosage form or a modified release dosage form (e.g., extended release, delayed release, multi-phasic release, sustained release). For example, in any embodiment, a solid oral dosage form comprising nafamostat or a pharmaceutically acceptable salt thereof, such as nafamostat mesylate, may be formulated as a plurality of controlled release beads where each bead includes a core, an active agent layer comprising the nafamostat (or salt thereof), and a controlled release layer comprising one or more polymers to provide a controlled release of the nafamostat after administration. Examples of suitable polymers include, but are not limited to, a cellulose ether (e.g. ETHOCEL®), an acrylate polymer or copolymer, a methacrylate polymer or copolymer, or a neutral or ionic (meth)acrylate-based polymer (e.g., EUDRAGIT® NE30D, RS, RL).

In another example, and in any embodiment, a solid oral dosage form comprising nafamostat or a pharmaceutically acceptable salt thereof, such as nafamostat mesylate, may be formulated as a plurality of controlled release beads as described above in combination with an immediate release component, such as a powder or granulate comprising nafamostat or a pharmaceutically acceptable salt thereof, such as nafamostat mesylate.

Solid oral dosage forms of nafamostat may include about 10 mg or more of nafamostat mesylate (or an equivalent amount of nafamostat free-base (about 4.5 mg or more) or other pharmaceutically acceptable salt), such as about 15 mg (about 6.8 mg freebase) or more, about 20 mg or more (about 9.0 mg or more of freebase), about 25 mg or more (about 11 mg or more of freebase), about 30 mg or more (about 13.6 mg or more of freebase), about 35 mg or more (about 16 mg or more of freebase), about 40 mg or more (about 14 mg or more of freebase), about 45 mg or more (about 20 mg or more of freebase), about 50 mg or more (about 23 mg or more of freebase), about 60 mg or more (about 27 mg or more of freebase), about 70 mg or more (about 32 mg or more of freebase), about 80 mg or more (about 36 mg or more of freebase), about 90 mg or more (about 41 mg or more of freebase), about 100 mg or more (about 45.2 mg or more of freebase), about 150 mg or more (about 67.9 mg or more of freebase), about 200 mg or more (about 90.5 mg or more of freebase), about 300 mg or more (about 136 mg or more of freebase), about 400 mg or more (about 181 mg or more of freebase), about 500 mg or more (about 226 mg or more of freebase), or about 600 mg or more (about 272 mg or more of freebase). A solid oral dosage form comprising nafamostat may include about 10 mg to about 600 mg of nafamostat mesylate (or an equivalent amount of nafamostat free-base (about 4.5 mg to about 271.5 mg ) or other pharmaceutically acceptable salt), such as about 50 mg to about 600 mg nafamostat mesylate, about 100 mg to about 600 mg, about 150 mg to about 600 mg, about 200 mg to about 600 mg, about 300 mg to about 600 mg, about 400 mg to about 600 mg, about 500 mg to about 600 mg, about 50 mg to about 500 mg, about 50 mg to about 400 mg, about 50 mg to about 300 mg, about 50 mg to about 200 mg, about 100 mg to about 500 mg, about 100 mg to about 400 mg, about 100 mg to about 300 mg, about 100 mg to about 200 mg, about 200 mg to about 500 mg, about 200 mg to about 400 mg, about 200 mg to about 300 mg, about 300 mg to about 500 mg, about 300 mg to about 400 mg, or about 400 mg to about 500 mg of nafamostat mesylate. In embodiments where the solid oral dosage form comprises a modified release component and an immediate release component, the total amount of nafamostat mesylate (or an equivalent amount of nafamostat free-base or other pharmaceutically acceptable salt) in the immediate release component may be about 1 mg to about 200 mg, such as about 2 mg to about 190 mg, about 3 mg to about 180 mg, about 4 mg to about 170 mg, about 5 mg to about 160 mg, about 6 mg to about 150 mg, about 7 mg to about 140 mg, about 8 mg to about 130 mg, about 9 mg to about 120 mg, or about 10 mg to about 100 mg nafamostat mesylate.

In any embodiment, a method for treating a viral infection in a subject comprising nafamostat (which may be included in an oral dosage form) may further comprise administering one or more additional one antiviral agents such as, but not limited to, oseltamivir (e.g., TAMIFLU™), zanamivir (e.g., RELENZA™), amantadine, rimantadine, remdesivir, chloroquine, ritonavir, lopinavir, ribavirin, penciclovir, nitazoxanide, nafamostat, favipiravir, corticosteroids, and combinations thereof. Examples of anti-malarial agents include atovaquone, chloroquine, doxycycline, mefloquine, primaquine, and tafenoquine. Examples of anti-HIV agents include emtricitabine, lamivudine, tenofovir disoproxil fumarate, and zidovudine.

In any embodiment, the subject to which nafamostat is administered orally may be any mammal. Examples of subjects include a primate, a human, a dog, a cat, a mouse, a rat, a cow, a horse, and a pig. In some examples, the subject is a human. The terms “subject,” “individual” or “patient” are used interchangeably and as used herein are intended to include human and non-human animals. Non-human animals include all vertebrates, for example, mammals and non-mammals, such as non-human primates, sheep, dogs, rats, cats, cows, horses, chickens, amphibians, and reptiles. Examples of mammals include non-human primates, sheep, dogs, cats, cows, and horses. In some examples, the subject is a human or humans. The methods are suitable for treating humans having a viral infection or disease. The subject may be symptomatic or asymptomatic with respect to the viral infection.

### Therapeutic Effects

A therapeutically effective amount of nafamostat, such as nafamostat mesylate, may be administered to a subject in need thereof prophylactically to prevent or inhibit a viral infection or retroactively to treat an ongoing viral infection.

Oral administration of a therapeutically effective amount of nafamostat to treat of an ongoing viral infection in a subject in need thereof may result in a clinically-relevant improvement in the subject’s condition with respect to the viral infection or disease caused there by achieving a therapeutically effective level of nafamostat in the GI tract, but not systemically (i.e., in the plasma), by having a Cₘₐₓ of below about 5 ng/mL. Oral administration of nafamostat, in contrast to systemic administration, is effectively a local administration to one or more mucosal membranes within the GI tract (e.g., the stomach, intestines, the anus). Therapeutically-relevant blood plasma levels were not observed in subjects administered oral nafamostat. Blood plasma levels achieved after intravenous administration of nafamostat range from about 5 mg/mL to 60 ng/mL for a 20 mg IV infusion to 70 ng/mL to 90 ng/mL for a 40 mg IV infusion (see, e.g., Okajima, K., et al., Cardiovascular Drug Reviews, Vol. 13, No. 1, pp. 51-65 or Cao, Y. et al, Biol. Pharm. Bull., Vol. 31, No. 11, pp. 1985-1989)). In contrast, blood plasma levels after oral administration of 200 mg nafamostat may be less than about 5 ng/mL, less than about 2 ng/mL, less than about 1 ng/mL, or a level below the limit of detection. However, despite the fact that nafamostat is administered locally and acting locally on the GI tract, the reduction in viral load in the GI tract reduces the overall viral load in the body, and therefore may also benefit systems outside the GI tract, such as the respiratory tract.

For example, oral administration of nafamostat may reduce or inhibit the progression of early-stages of infection to late stage respiratory distress. Complications of COVID-19 include development of acute respiratory distress syndrome (ARDS) and cytokine release syndrome (CRS). Oral administration of nafamostat according to the methods described herein may avoid progression to one or both of ARDS and CRS or may ameliorate or reduce the symptoms thereof. Oral administration of a therapeutically effective amount of nafamostat may improve symptoms of an ongoing viral infection, such as increasing blood-oxygen levels, body temperature, nausea, diarrhea, vomiting, fatigue, congestion, loss of taste, loss of smell, ability to breath, chest pain, confusion, or any other adverse symptom associated with COVID-19, when compared to a control non-treated population. Oral administration of a therapeutically effective amount of nafamostat may decrease gut viral concentration/load and therefore may cause a reduction in systemic viral concentration/load when compared to a control non-treated population. Nafamostat may be administered in an amount to effectively inhibit viral infection in the subject, for example, inhibit the ability of a virus to enter a host cell. For example, a method of treating a viral infection in a subject may comprise orally administering a therapeutically effective amount of nafamostat as described herein to the subject, wherein the therapeutically effective amount is any as described above (e.g., about 50 mg to about 500 mg nafamostat mesylate per administration (or an equivalent amount of freebase or other pharmaceutically acceptable salt), such as 100 mg, 200 mg, 300 mg, 400 mg, or any range there between or about 150 mg per day to about 1500 mg per day, or any range there between). In any embodiment, the administering may comprise administering a solid oral dosage form comprising nafamostat mesylate, nafamostat freebase, or another pharmaceutically acceptable salt of nafamostat.

Oral administration of a therapeutically effective amount of nafamostat to prophylactically prevent or inhibit a viral infection in a subject may result in circumvention of viral infection or a viral infection that is less severe than would otherwise have occurred without the use of orally administered nafamostat. Nafamostat may be administered in an amount to effectively inhibit viral infection within the subject. For example, a method of preventing or inhibiting a viral infection in a subject may comprise orally administering a therapeutically effective amount of nafamostat, wherein the therapeutically effective amount is any as described above (e.g., about 50 mg to about 500 mg nafamostat mesylate (or an equivalent amount of nafamostat free base or other pharmaceutically acceptable salt) per administration, or any range there between or about 150 mg to about 1500 mg nafamostat mesylate per day, or any range there between). In any embodiment, the administering may comprise administering a solid oral dosage form comprising nafamostat or a pharmaceutically acceptable salt thereof, such as nafamostat mesylate. As will be discussed further in the examples below, no adverse events are observed when nafamostat is administered orally in an amount of up to 600 mg nafamostat mesylate. The LD₅₀ for a human is expected to be much higher, such as 2000 mg, 3000 mg, or even higher. Nafamostat is therefore uniquely poised as a potentially prophylactic agent to prevent viral infection (such as a coronaviral infection), which may be particularly beneficial during a pandemic, such as the SARS-CoV-2 pandemic, which at the time of this filing, is ongoing. A prophylactic approach may help stop the spread of the virus between individuals and also to reduce viral load in an infected individual and reduce incidence of severe complications associated with the infection.

Sub-populations that may be administered oral nafamostat on a prophylactic basis for inhibiting a viral infection include those identified as high-risk for developing severe symptoms if infected with SARS-CoV-2. High-risk conditions include but are not limited to, an age of 65 or greater, cancer, chronic kidney disease, chronic obstructive pulmonary disease (COPD), down syndrome, heart failure, coronary artery disease, cardiomyopathy, sickle cell disease, type II diabetes mellitus, a BMI of 30 kg/m² or higher, an immunocompromised condition from organ transplant, pregnancy, a smoking habit, inflammatory bowel disease (IBD), and irritable bowel syndrome (IBS). Other high-risk conditions include but are not limited to, moderate-to-severe asthma, cerebrovascular disease, cystic fibrosis, hypertension, liver disease, dementia, Alzheimer’s Disease, pulmonary fibrosis, thalassemia, type I diabetes mellitus, a BMI of 25 kg/m² to 30 kg/m², and an immunocompromised condition from blood or bone marrow transplant, immune deficiency, HIV, other digestive conditions (other than IBD and IBS), and use of corticosteroid or other immune-weakening medicine.

Other sub-populations that may be administered oral nafamostat on a prophylactic basis for inhibiting a viral infection include those at higher risk for exposure to the virus, such as, but not limited to, healthcare workers (e.g., nurses, doctors, pharmacists and pharmacy technicians, EMS personnel, healthcare assistants, dentists and dental hygienists, therapists, phlebotomists, chiropractors, contractual healthcare workers), long-term care facility residents and personnel, education workers (e.g., teachers, school staff, school bus drivers), public transit workers, clergy, early childhood caregivers, adult caregivers, manufacturing workers, postal service workers (and other delivery service providers), food and agricultural workers, grocery store workers, correctional officers and other essential employees in a congregate setting, and anyone who is regularly exposed to a congregate setting or who live in close quarters and cannot socially distance. Inhibition of a viral infection in any individual in the aforementioned groups will likewise prevent infection in other individuals around them in these settings.

Various symptoms of a viral infection may be measured quantitatively and therefore improvement in a subject’s condition in response to a method of treatment, as described herein, may be assessed in a quantitative manner as well. For example, oral administration of a therapeutically effective amount of nafamostat may result in one or more of a reduction in viral load (systemic, gut, pulmonary, fecal, and the like), reduction in white blood count, reduction in cytokine levels (e.g., of one or more of TNF-α, IL-1β, IL-6, IL-8, IFNy, IL-17, IL-18, IL-1α, and IL-1RA), and an increase in blood oxygen levels. For example, a reduction of at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, and in an ideal situation, about 100% reduction (complete elimination of disease, symptom, virus concentration, or other undesired property) may be achieved for these or any other quantitatively measured markers of viral infection/disease severity.

Efficacy of nafamostat may be measured based on a Time to Clinical Improvement (TTCI), measured in days from initial treatment until an increase in at least two categories on the following ordinal scale of clinical status: 1) death; 2) hospitalized on invasive mechanical ventilation or extracorporeal membrane oxygenation (ECMO); 3) hospitalized, on non-invasive ventilation or high-flow oxygen device; 4) hospitalized, requiring supplemental oxygen 5) hospitalized, not requiring supplemental oxygen but requiring ongoing medical care; 6) hospitalized, not requiring supplemental oxygen or ongoing medical care; 7) not hospitalized, limited in activity or requiring home oxygen; 8) not hospitalized and no limitation on activity. Efficacy of nafamostat may also be measured, for example, by a reduction of time to hospital discharge, maintaining a NEWS2 score of 2 or lower for 24 hours, decreased mortality across nafamostat-treated patients, decreased duration of mechanical ventilation, decreased duration of ECMO, decreased duration of supplemental oxygen, a reduction in length of hospital stay, a reduction in time to reach undetectable viral levels in upper and lower respiratory samples, and a reduction in viral levels in upper and lower respiratory samples. Other suitable metrics include but are not limited to, reduced chest/throat congestion, reduced pain or discomfort, reduced breathing difficulties, and reduced fatigue, any of which may be perceived by a healthcare professional, caretaker, or may be self-reported by the subject. A similar scale may be developed pertaining more particularly to GI symptoms associated with coronavirus infection, such as reduction in the severity or frequency of one or more of vomiting, nausea, and diarrhea.

### Adverse Events (AEs)

Currently, nafamostat is approved for intravenous, intra-arterial, or extracorporeal administration, each of which introduces the drug into systemic circulation, which always carries a risk of causing various non-specific and off-target effects. Indeed, adverse reactions that have been reported with the use of intravenously-delivered nafamostat include allergic reaction, diabetic ketoacidosis, agranulocytosis, and hyperkalemia. While these reactions are rare, avoiding systemic administration of a drug when systemic administration is not entirely necessary represents a lower risk to a subject. Oral administration of nafamostat, as described herein, avoids substantive introduction of nafamostat into systemic circulation. No nafamostat was detected in blood samples, that indicates that either a) no nafamostat entered systemic administration or b) only a small amount of nafamostat entered the blood stream. In the latter case, even if a small amount would enter the blood stream through GI membranes, it would be rapidly metabolized by serum proteases and not pose a high risk for off-target effects. Therefore, the present disclosure also provides a method of treating a viral infection in a subject in need thereof comprising orally administering a therapeutically effective amount of nafamostat or a pharmaceutically acceptable salt thereof (e.g., mesylate) to the subject, wherein the incidence of adverse events related to off-target effects of nafamostat are reduced when compared to the incidence of adverse effects experience after intravenous administration of nafamostat. Examples of adverse effects include but are not limited to, allergic reaction, diabetic ketoacidosis, agranulocytosis, and hyperkalemia. Advantageously, even though the nafamostat is acting locally in the GI tract to reduce viral load, the benefits may be observed elsewhere in the body, similar to the benefits of a systemic administration. The methods described herein may be able to provide whole-body benefits in treating a viral infection even while limiting administration and action to a local level.

Definitions and Terminology: The following definitions and description applies to any and all embodiments and aspects of the invention as described herein unless explicitly indicated otherwise.

As used herein, the term “about” when immediately preceding a numerical value means a range of plus or minus 10% of that value unless the context of the disclosure indicates otherwise, or is inconsistent with such an interpretation. For example, “about 50” means 45 to 55 and “about 25,000” means 22,500 to 27,500.

As used in this disclosure, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

“Comprising” is used herein to mean “including, but not limited to.” While various compositions, methods, and devices are described in terms of “comprising” various components or steps, in any embodiment the composition or method can also “consist essentially of” or “consist of” the described components and steps, and such terminology should be interpreted as defining essentially closed-member groups. Other terms that are used herein to indicate “including, but not limited to” are “including,” “having,” “have,” “contain,” and the like. Similarly, the term “having” should be interpreted as “having at least,” the term “includes” should be interpreted as “includes, but is not limited to”).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C,” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together). In instances where a convention analogous to “at least one of A, B, and C” is used, such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

This disclosure is not limited to the particular systems, devices and methods described, as these may vary. The terminology used in the description is for the purpose of describing the particular versions or embodiments only, and is not intended to limit the scope. Variations of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

### Embodiments

The disclosure provides the following non-limiting embodiments:

Embodiment 1 comprises a method of treating a viral infection in a subject comprising orally administering a therapeutically effective amount of nafamostat or a pharmaceutically acceptable salt thereof to the subject in need thereof, wherein the amount of nafamostat measurable in the plasma of the subject after 1 to 8 hours after administering is below about 5 ng/mL.

Embodiment 2 comprises the method of Embodiment 1, wherein the viral infection comprises infection by coronavirus, SARS-CoV, MERS-CoV, SARS-CoV-2, or another mutated coronavirus.

Embodiment 3 comprises the method of Embodiment 1or 2, wherein the viral infection comprises infection by a coronavirus.

Embodiment 4 comprises the method of any one of Embodiments 1-3, wherein the therapeutically effective amount of nafamostat is equivalent to 100 mg to about 1500 mg of nafamostat mesylate.

Embodiment 5 comprises the method of any one of Embodiments 1-4, wherein the effective amount of nafamostat or a pharmaceutically acceptable salt thereof is administered to the subject as one or more solid oral dosage forms comprising the nafamostat or a pharmaceutically acceptable salt thereof.

Embodiment 6 comprises the method of Embodiment 5, wherein the solid oral dosage form is a tablet, a bi-layer tablet, a capsule, a multiparticulate, a drug-coated sphere, a matrix tablet, or a multicore tablet.

Embodiment 7 comprises the method of Embodiment 5 or 6, wherein the solid oral dosage form comprises about 50 mg to about 200 mg of nafamostat mesylate or an equivalent amount of nafamostat or a pharmaceutically acceptable salt thereof.

Embodiment 8 comprises the method of any one of Embodiments 1-7, wherein the subject is a mammal.

Embodiment 9 comprises the method of any of Embodiments 1-8, wherein the subject is a human.

Embodiment 10 comprises the method of any of Embodiments 1-9, wherein the administering comprises administering nafamostat in an amount equivalent to about 50 mg to about 500 mg nafamostat mesylate once, twice, or three times per day to the subject.

Embodiment 11 comprises the method of any of Embodiments 1-10, wherein the administering comprises administering nafamostat in an amount equivalent to at least about 0.1 mg nafamostat mesylate per kg of the subject’s weight per day.

Embodiment 12 comprises the method of any of Embodiments 1-11, wherein the administering comprises administering nafamostat in an amount equivalent to about 0.75 mg nafamostat mesylate per kg of the subject’s weight per day to about 20 mg per kg of the subject’s weight per day.

Embodiment 13 comprises the method of any of Embodiments 1-12, wherein the administering comprises administering nafamostat in an amount equivalent to about 1 mg nafamostat mesylate per kg of the subject’s weight per day to about 10 mg nafamostat mesylate per kg of the subject’s weight per day.

Embodiment 14 comprises the method of any of Embodiments 1-13, wherein the administering is self-administering.

Embodiment 15 comprises the method of any of Embodiments 1-14, wherein the administering is performed at a location that is not in a hospital.

Embodiment 16 comprises a method of inhibiting a viral infection in a subject, the method comprising orally administering a therapeutically effective amount of nafamostat or a pharmaceutically acceptable salt thereof.

Embodiment 17 comprises the method of Embodiment 16, wherein the administering occurs before a symptom of a viral infection is observed in the subject.

Embodiment 18 comprises the method of Embodiment 16 or Embodiment 17, wherein the subject has been exposed to a virus.

Embodiment 19 comprises the method of any one of Embodiments 16-18, wherein the viral infection comprises infection by a coronavirus, SARS-CoV, MERS-CoV, SARS-CoV-2, or another mutation of a coronavirus.

Embodiment 20 comprises the method of any one of Embodiments 16-19, wherein the viral infection comprises infection by a coronavirus.

Embodiment 21 comprises the method of any one of Embodiments 16-20, wherein the subject is aged 65 or older.

Embodiment 22 comprises the method of any one of Embodiments 16-21, wherein the subject has one or more of cancer, chronic kidney disease, chronic obstructive pulmonary disease (COPD), down syndrome, heart failure, coronary artery disease, cardiomyopathy, sickle cell disease, type II diabetes mellitus, a BMI of 30 kg/m² or higher, an immunocompromised condition from organ transplant, pregnancy, a smoking habit, inflammatory bowel disease, and irritable bowel syndrome.

Embodiment 23 comprises the method of any one of Embodiments 16-22, wherein the subject has one or more of moderate-to-severe asthma, cerebrovascular disease, cystic fibrosis, hypertension, liver disease, dementia, Alzheimer’s Disease, pulmonary fibrosis, thalassemia, type I diabetes mellitus, a BMI of 25 kg/m² to 30 kg/m², and an immunocompromised condition from blood or bone marrow transplant, immune deficiency, HIV, or use of corticosteroid or other immune-weakening medicine.

Embodiment 24 comprises the method of any one of Embodiments 16 to 23, wherein the administering comprising administering one or more solid oral dosage forms comprising nafamostat or a pharmaceutically acceptable salt of nafamostat.

Embodiment 25 comprises the method of Embodiment 24, wherein the solid oral dosage form is a tablet, a bi-layer tablet, a capsule, a multiparticulate, a drug-coated sphere, a matrix tablet, or a multicore tablet.

Embodiment 26 comprises the method of Embodiment 24 or Embodiment 25, wherein the solid oral dosage form comprises about 50 mg to about 600 mg of nafamostat mesylate or an equivalent amount of nafamostat or a pharmaceutically acceptable salt thereof.

Embodiment 27 comprises the method of any of Embodiments 16-26, wherein the subject is a mammal.

Embodiment 28 comprises the method of any of Embodiments 16-27, wherein the subject is a human.

Embodiment 29 comprises the method of any of Embodiments 16-28, wherein the administering comprises administering nafamostat in an amount equivalent to about 200 mg to about 500 mg nafamostat mesylate once, twice, or three times per day to the subject.

Embodiment 30 comprises the method of any of Embodiments 16-29, wherein the administering comprises administering nafamostat in an amount equivalent to at least about 0.1 mg nafamostat mesylate per kg of the subject’s weight per day.

Embodiment 31 comprises the method of any of Embodiments 16-30, wherein the administering comprises administering nafamostat in an amount equivalent to about 0.75 mg nafamostat mesylate per kg of the subject’s weight per day to about 20 mg nafamostat mesylate per kg of the subject’s weight per day.

Embodiment 32 comprises the method of any of Embodiments 16-31, wherein the administering comprises administering nafamostat in an amount equivalent to about 1 mg per kg of the subject’s weight per day to about 10 mg per kg of the subject’s weight per day.

Embodiment 33 comprises the method of any of Embodiments 16-32, wherein the administering is self-administering.

Embodiment 34 comprises the method of any of Embodiments 16-33, wherein the administering is performed at a location that is not in a hospital.

Embodiment 35 comprises a method of reducing the risk of experiencing an adverse event associated with intravenous administration of nafamostat mesylate in a subject having a viral infection comprising orally administering a therapeutically effective amount of nafamostat mesylate to the subject, wherein the adverse event is one or more of allergic reaction, diabetic ketoacidosis, agranulocytosis, and hyperkalemia.

Embodiment 36 comprises the method of Embodiment 35, wherein the administering comprising administering one or more solid oral dosage forms comprising nafamostat mesylate.

Embodiment 37 comprises the method of Embodiment 35 or Embodiment 36, wherein the solid oral dosage form is a tablet, a bi-layer tablet, a capsule, a multiparticulate, a drug-coated sphere, a matrix tablet, or a multicore tablet.

Embodiment 38 comprises the method of any one of Embodiments 1-37, wherein the administering of the nafamostat or pharmaceutically acceptable salt thereof reduces a viral load of the virus in the GI tract of the subject.

Embodiment 38 comprises the method of any one of Embodiments 1-38, wherein the administering of the nafamostat or pharmaceutically acceptable salt thereof reduces a systemic viral load of the virus in the subject.

Embodiment 38 comprises the method of any one of Embodiments 1-38, wherein the administering of the nafamostat or pharmaceutically acceptable salt thereof reduces the plasma levels of at least one cytokine selected from TNF-α, IL-1β, IL-6, IL-8, IFNy, IL-17, IL-18, IL-1α, and IL-1RA.

### EXAMPLES

Example 1: Pharmacokinetic Studies. The pharmacokinetics of nafamostat were evaluated in male rats following an intravenous dose of 1 mg/kg or an oral dose of 9 mg/kg or 10 mg/kg. Nafamostat whole blood concentrations were determined by LC/MS/MS. Following intravenous dosing, nafamostat appeared in the blood of rats, with average (± SD) maximum whole blood concentrations (189 ± 54 ng/mL) being reached at the first measured time point (0.0830 hours) in all animals. Whole blood concentrations declined thereafter with a terminal t_{½} of 1.84 ± 1.3 hours. The clearance of nafamostat was high and greater than hepatic blood flow in rats. The AUC₀₋ₜ was 81.8 ± 29 ng̬·h/mL. The pharmacokinetic parameters were generated using a standard, non-compartmental model. Note that the half-life reported in this section is the terminal half-life and the alpha phase half-life is much shorter (estimated to be about 3 minutes).

After oral dosing, nafamostat appeared in the blood for the majority of the rats, with the whole blood concentrations being low and transient. The average (± SD) maximum plasma concentrations were 6.40 ± 6.2 ng/mL and 6.09 ± 3.6 ng/mL for the 9 mg/kg and 10 mg/kg doses, respectively, being reached between 0.250 hours and 0.500 hours. Due to the transient whole blood concentration-time profiles, the terminal t_{½} could not be calculated in the majority of the subjects but is approximated to be about 3 hours. The AUC₀₋ₜ was 8.80 ng·h/mL (n=1) and 12.6 ± 11 ng·h/mL (n=3) for the two doses, respectively. The overall absolute oral bioavailability of nafamostat was calculated as 1.16 ± 1.1% in rats.

Example 2: Safety and Tolerability Studies in Animals. Several in vitro assays and in vivo studies have been conducted to evaluate the exploratory safety of nafamostat mesylate. The effects of nafamostat on hERG K⁺ channel current and the cytotoxic and mutagenic potential of nafamostat were studied. The in vivo studies included pilot non-GLP single and repeat-dose oral toxicity range-finding studies of nafamostat in SD rats. The following in vitro tests were conducted:

Effects of nafamostat in an hERG assay: In an in vitro non-GLP screening study, effects of nafamostat on the hERG K⁺ channel currents were evaluated in voltage-clamped human embryonic kidney cells at room temperature. Nafamostat was tested in 3 cells at each of 4 concentrations from 0.1 µM to 50 µM. Nafamostat mesylate inhibited hERG potassium currents by approximately 24% at 50 µM. The positive control compound E-4031 inhibited hERG potassium currents by approximately 99% at 0.5 µM, confirming the sensitivity of the test system. The IC₅₀ of nafamostat on hERG potassium currents was estimated as > 50 µM, indicating a weak response and low potential for effects on the QT interval. A full ICH-Compliant hERG study and in vivo cardiovascular safety pharmacology study is be performed as part of the IND-enabling program.

Bacterial Reverse Mutation (Ames) Screening Assay: This in vitro non-GLP study evaluated the cytotoxic and mutagenic activity of nafamostat in S. typhimurium strains TA98, TA100, TA1535, and TA97a and in the Escherichia coli (E. coli) strain WP2uvrA76. Two independent assays were conducted using the plate incorporation method with and without S9 metabolic activation. The highest concentration tested was 25 mg/mL. The vehicle control, sterile saline, was tested under the same conditions. Concurrent positive controls evaluated under the same conditions demonstrated the sensitivity of the assay and the metabolizing activity of the S9 mix. Nafamostat was not cytotoxic and no positive increases in the mean number of revertants for each plate were observed in any test strains with or without S9 at concentrations up to 25 mg/mL.

The following in vivo tests were conducted:

Oral Tolerability in Rats: The single dose tolerability to oral nafamostat was studied in SD rats in a non-GLP pilot dose range-finding study. A single oral dose of nafamostat mesylate (300, 600, 800 and 1000 mg/kg) was administered by oral gavage to 3 males and 3 females per dose group and the animals were observed for 7 days and then sacrificed and necropsies performed. There were no abnormalities observed at doses up to 800 mg/kg. In the 1000 mg/kg female dose group, 2 of 3 animals died at 48 hours, but upon necropsy both were observed to have perforations in the stomach indicating dosing misadministration. All other animals survived to 7 days and had no abnormalities at necropsy. The results suggest a single dose oral tolerability of 1000 mg/kg or greater, which agrees with published results (Sato K, Watanabe K, Terasawa K, Yokomoto Y, Nagai N, Otani K., Acute Toxicity Studies of FUT-175 (Nafamostat mesilate) in Mice and Rats. Nippon Yakurigaku Zasshi 1984; 18(8):227-234) indicating a LD₅₀ in mice of 4600 to 5190 mg/kg and in rat of 2750 to 3050 mg/kg.

A pilot, non-GLP repeat-dose study was performed in which nafamostat mesylate (500 or 1000 mg/kg/day PO) was administered to 6 male SD rats per group for 5 days. Four of 6 animals receiving 1000 mg/kg/day died by day 5 and were observed to have fluid filled stomach at necropsy. One animal had petechiae on the lower third of the colon. No other gross abnormalities were noted at necropsy. Death in 2 of the animals followed seizure activity at about 1 h post-dose. All animals in the 500 mg/kg/day group survived five days of dosing and were sacrificed on Day 7. There were no abnormal findings at necropsy.

### Example 2: Investigation of Safety, Tolerability, and Pharmacokinetics of Sequential Dose Regimens of Oral Nafamostat Mesylate in Healthy Male and Female Subjects.

Part 1: Single Ascending Dose Study. Male and female subjects aged 18 - 70 were administered a single dose of 50 mg nafamostat mesylate on day 1, 100 mg nafamostat mesylate on day 3 or 4, and 200 mg nafamostat mesylate on day 5, 6, or 7. Nafamostat mesylate was administered in the form of an oral solution. Blood samples were obtained at pre-dose (0 hr.) and at 0.5, 1, 2, 3, 4, 6, 8, 12, and 24 hours after receiving the nafamostat mesylate oral solution.

Part 2: Multiple Ascending Dose Study. Subjects were divided into 2 cohorts. Cohort 1 received an oral solution of 100 mg/mL nafamostat mesylate twice a day for 5 days with pharmacokinetic evaluations on each day. Blood samples were obtained at pre-dose (0 hr.) and at 0.5, 1, 2, 3, 4, 6, 8, 12, and 24 hours after receiving the nafamostat mesylate oral solution as well as after the first dose on Day 5. Safety evaluations continued for 48 hours after the last dose. After review and evidence of safety, Cohort 2 received an oral solution of 200 mg/mL nafamostat mesylate twice a day for 5 days with pharmacokinetic evaluations on each day. Blood samples were obtained at pre-dose (0 hr.) and at 0.5, 1, 2, 3, 4, 6, 8, 12, and 24 hours after receiving the first dose of nafamostat mesylate oral solution as well as after the first dose on Day 5. Safety evaluations continued for 48 hours after the last dose.

Part 3: Single Fixed Dose Administered Twice Daily for 5 Days. Six subjects, different than the subjects from Parts 1 and 2, received an oral solution of 200 mg/mL nafamostat mesylate twice a day for 5 days. Blood samples were obtained at pre-dose (0 hr.) and at 0.5, 1, 2, 3, 4, 6, and 8 hours after the first dose on Day 1 and again after the first dose on Day 5.

Part 4: Single Ascending Dose Study. Three subjects were administered a single dose of an oral solution of 400 mg/mL nafamostat mesylate. After review and evidence of safety, a second set of three subjects received a single dose of an oral solution of 600 mg/mL nafamostat mesylate. Blood samples were obtained for pharmacokinetic analysis at pre-dose (0 hr.) and at 0.25, 0.5, 0.75, 1, 1.5, 2, 2.5, 3, 4, 6, and 8 hours after receiving the nafamostat mesylate dose.

Inclusion criteria for subjects included a normal blood pressure and heart rate without medication; a clinical chemistry profile including electrolytes, alkaline phosphatase, lactate dehydrogenase, creatinine phosphokinase (CPK), creatinine, and urea within the normal range without medication; liver enzyme levels not more than 1.5 times the normal range; CPK levels not more than 2 times the normal range; normal urinary creatinine levels; abstinence from smoking; not pregnant or lactating; no use of chemotherapy agents or history of cancer within 5 years; no use of antibiotic or antiviral within 1 month; no history of congestive heart failure; no use of P450 inducers or inhibitors within 30 days; no use of dietary aids or foods known to modulate drug metabolizing enzymes within 14 days; easily able to swallow oral medications; no history of seizure disorder; no use of prescription drugs (excluding hormonal birth control) within 1 month, and no use of over the counter medications (excluding non-mega-dose vitamins) within 1 week.

Oral solutions received by each subject were prepared by dissolving nafamostat mesylate in about 100 mL of EMETROL®/water and provided at a volume to deliver the indicated dosage. There was no placebo formulation.

Evaluations of subjects included incidence and severity of adverse events (AEs), vital signs (pulse rate, blood pressure, respiratory rate), 12-lead electrocardiogram, electrocardiogram telemetry or MCOT cardiac patch, continuous pulse oximetry, clinical laboratory tests (CBC-diff, complete metabolic panel including LFTs, PT, PTT), physical examination.

Nafamostat pharmacokinetics was evaluated at the 200 mg dose levels. As shown below in Table 1, except for three samples (one of which was at 0 hours), all PK measurements were below the lower limit of quantitation (LLQ; 0.1 ng/mL), therefore demonstrating the limited systemic bioavailability of nafamostat. The two samples that were outliers and were taken after at least one daily dose for the day were found to have 0.125 ng/mL and 0.101 ng/mL nafamostat.

TABLE 1

| Subject | Hour (post-dose) | Day | NM (ng/mL) | Day | NM (ng/mL) | Day | NM (ng/mL) | Day | NM (ng/mL) |
|---|---|---|---|---|---|---|---|---|---|
| 12 | 0 | 1 | < 0.100 | 2 | < 0.100 | 3 | < 0.100 | 4 | < 0.100 |
| 12 | 0.5 | 1 | < 0.100 | 2 | < 0.100 | - | - | - | - |
| 12 | 1 | 1 | < 0.100 | 2 | < 0.100 | - | - | - | - |
| 12 | 2 | 1 | < 0.100 | 2 | < 0.100 | - | - | - | - |
| 12 | 3 | 1 | < 0.100 | 2 | < 0.100 | - | - | - | - |
| 12 | 4 | 1 | < 0.100 | 2 | < 0.100 | 3 | < 0.100 | 4 | < 0.100 |
| 12 | 6 | 1 | < 0.100 | 2 | < 0.100 | - | - | - | - |
| 12 | 8 | 1 | < 0.100 | 2 | < 0.100 | 3 | < 0.100 | - | - |
| 12 | 12 | 1 | < 0.100 | 2 | < 0.100 | 3 | < 0.100 | - | - |
| 12 | 16 | - | - | 2 | < 0.100 | 3 | < 0.100 | - | - |
| 13 | 0 | 1 | < 0.100 | 2 | < 0.100 | 3 | < 0.100 | 4 | < 0.100 |
| 13 | 0.5 | 1 | < 0.100 | 2 | < 0.100 | - | - | - | - |
| 13 | 1 | 1 | < 0.100 | 2 | < 0.100 | - | - | - | - |
| 13 | 2 | 1 | < 0.100 | 2 | < 0.100 | - | - | - | - |
| 13 | 3 | 1 | < 0.100 | 2 | < 0.100 | - | - | - | - |
| 13 | 4 | 1 | < 0.100 | 2 | < 0.100 | 3 | < 0.100 | 4 | < 0.100 |
| 13 | 6 | 1 | < 0.100 | 2 | < 0.100 | - | - | - | - |
| 13 | 8 | 1 | < 0.100 | 2 | < 0.100 | 3 | < 0.100 | - | - |
| 13 | 12 | 1 | < 0.100 | 2 | < 0.100 | 3 | < 0.100 | - | - |
| 13 | 16 | - | - | 2 | < 0.100 | 3 | < 0.100 | - | - |
| 14 | 0 | 1 | < 0.100 | 2 | < 0.100 | 3 | < 0.100 | 4 | < 0.100 |
| 14 | 0.5 | 1 | < 0.100 | 2 | < 0.100 | - | - | - | - |
| 14 | 1 | 1 | < 0.100 | 2 | < 0.100 | - | - | - | - |
| 14 | 2 | 1 | < 0.100 | 2 | < 0.100 | - | - | - | - |
| 14 | 3 | 1 | < 0.100 | 2 | < 0.100 | - | - | - | - |
| 14 | 4 | 1 | < 0.100 | 2 | < 0.100 | 3 | 0.101 | 4 | < 0.100 |
| 14 | 6 | 1 | < 0.100 | 2 | < 0.100 | - | - | - | - |
| 14 | 8 | 1 | < 0.100 | 2 | < 0.100 | 3 | < 0.100 | - | - |
| 14 | 12 | 1 | < 0.100 | 2 | < 0.100 | 3 | < 0.100 | - | - |
| 14 | 16 | - | - | 2 | < 0.100 | 3 | < 0.100 | - | - |
| 15 | 0 | 1 | < 0.100 | 2 | < 0.100 | 3 | < 0.100 | 4 | < 0.100 |
| 15 | 0.5 | 1 | < 0.100 | 2 | < 0.100 | - | - | - | - |
| 15 | 1 | 1 | < 0.100 | 2 | < 0.100 | - | - | - | - |
| 15 | 2 | 1 | < 0.100 | 2 | < 0.100 | - | - | - | - |
| 15 | 3 | 1 | < 0.100 | 2 | < 0.100 | - | - | - | - |
| 15 | 4 | 1 | < 0.100 | 2 | < 0.100 | 3 | < 0.100 | 4 | < 0.100 |
| 15 | 6 | 1 | < 0.100 | 2 | < 0.100 | - | - | - | - |
| 15 | 8 | 1 | < 0.100 | 2 | < 0.100 | 3 | < 0.100 | - | - |
| 15 | 12 | 1 | < 0.100 | 2 | < 0.100 | 3 | < 0.100 | - | - |
| 15 | 16 | - | - | 2 | < 0.100 | 3 | < 0.100 | - | - |
| 16 | 0 | 1 | < 0.100 | 2 | < 0.100 | 3 | < 0.100 | 4 | < 0.100 |
| 16 | 0.5 | 1 | < 0.100 | 2 | < 0.100 | - | - | - | - |
| 16 | 1 | 1 | 0.125 | 2 | < 0.100 | - | - | - | - |
| 16 | 2 | 1 | < 0.100 | 2 | < 0.100 | - | - | - | - |
| 16 | 3 | 1 | < 0.100 | 2 | < 0.100 | - | - | - | - |
| 16 | 4 | 1 | < 0.100 | 2 | < 0.100 | 3 | < 0.100 | 4 | < 0.100 |
| 16 | 6 | 1 | < 0.100 | 2 | < 0.100 | - | - | - | - |
| 16 | 8 | 1 | < 0.100 | 2 | < 0.100 | 3 | < 0.100 | - | - |
| 16 | 12 | 1 | < 0.100 | 2 | < 0.100 | 3 | < 0.100 | - | - |
| 16 | 16 | - | - | 2 | < 0.100 | 3 | < 0.100 | - | - |
| 17 | 0 | 1 | < 0.100 | 2 | < 0.100 | 3 | < 0.100 | 4 | 0.457 |
| 17 | 0.5 | 1 | < 0.100 | 2 | < 0.100 | - | - | - | - |
| 17 | 1 | 1 | < 0.100 | 2 | < 0.100 | - | - | - | - |
| 17 | 2 | 1 | < 0.100 | 2 | < 0.100 | - | - | - | - |
| 17 | 3 | 1 | < 0.100 | 2 | < 0.100 | - | - | - | - |
| 17 | 4 | 1 | < 0.100 | 2 | < 0.100 | 3 | < 0.100 | 4 | < 0.100 |
| 17 | 6 | 1 | < 0.100 | 2 | < 0.100 | - | - | - | - |
| 17 | 8 | 1 | < 0.100 | 2 | < 0.100 | 3 | < 0.100 | - | - |
| 17 | 12 | 1 | < 0.100 | 2 | < 0.100 | 3 | < 0.100 | - | - |
| 17 | 16 | - | - | 2 | < 0.100 | 3 | < 0.100 | - | - |

There were no adverse events that could be attributed to nafamostat reported, as shown below in Table 2.

TABLE 2

| Subject | Adverse Event? | Adverse Event | Severity | Drug-Related? | Resolved? |
|---|---|---|---|---|---|
| 12 | Y | Intermittent headache | Mild | No | Y |
| 13 | Y | Thrush | Moderate | No | Y |
| 14 | Y | Tonsillitis right | Mild | No | Y |
| 14 | Y | Right thigh redness, swelling | Mild | No | Y |
| 15 | Y | Nausea | Moderate | No | Y |
| 15 | Y | Orthostatic tachycardia* * | Moderate | No | N |
| 16 | N | - | - | - | - |
| 17 | Y | Chest pain (mild, intermittent) | Mild | No | Y |

** Part 2, day 6 (discharge day)

Example 3: A Phase 2 Adaptive, Randomized, Double-Blind Study to Determine the Efficacy, Safety, and Tolerability of Orally Administered Nafamostat Mesylate in Adult Subjects Admitted to Hospital due to COVID-19. Up to 60 subjects are randomized using an adaptive statistical design. Subjects are hospitalized for COVID-19 and undergo medical screening to determine eligibility for the study. Two-thirds of the subjects (Cohort 1) receive an oral nafamostat mesylate solution twice a day for 10 days and one-third of subjects (Cohort 2) receive an oral placebo solution twice a day for 10 days. Cohort 1 is divided into two sub-cohorts, each receiving a different dose of nafamostat mesylate (up to a maximum of 200 mg, twice a day).

Subject inclusion criteria include an age of 18 to 99, having been admitted to the hospital due to signs and symptoms of COVID-19, a positive test for SARS-CoV-2, and at least one of a) radiographic infiltrates by imaging, b) clinical assessment (evidence of rales/crackles on exam) and an SpO₂ of 94% or less on room air, and c) requiring supplemental oxygen. Exclusion criteria include intubation, inability to swallow an oral solution, positive serology for HIV or Hepatitis (B or C), an ALT/AST greater than 5 times the upper normal limit, pregnancy or lactation, an estimated glomerular filtration rate (eGFR) of less than 50 or requiring dialysis, and a known allergy to nafamostat or related drugs (e.g., other protease inhibitors).

Nafamostat mesylate is dissolved in about 100 mL of EMETROL®/water and are delivered orally twice a day to each subject for up to 10 days. Placebo consists of about 100 mL of orally administered EMETROL®/water twice a day to each subject for up to 10 days.

Efficacy is measured by the percentage of subjects reporting severity of condition based on an ordinal clinical scale on Day 10. The ordinal scale is developed by the WHO and assesses the clinical status at the first assessment of a given day, has been modified for the current study, and is as follows: 1) death; 2) hospitalized on invasive mechanical ventilation or extracorporeal membrane oxygenation (ECMO); 3) hospitalized, on non-invasive ventilation or high-flow oxygen device; 4) hospitalized, requiring supplemental oxygen 5) hospitalized, not requiring supplemental oxygen but requiring ongoing medical care; 6) hospitalized, not requiring supplemental oxygen or ongoing medical care; 7) not hospitalized. Efficacy is also measured on other aspects of the ordinal scale (e.g., daily ordinal scale scores, change in ordinal scale scores, time of improvement in any particular category, time to improvement in any particular two categories); change of NEWS2 score from baseline; time to discharge; time to a NEWS2 score of two or less that is maintained for at least 24 hours; mean time to hospital discharge order; mortality; incidence of Grade 3 and Grade 4 AEs; incidence of serious AEs (SAEs); use of oxygen or ventilation (e.g., incidence of and mean duration of new non-invasive ventilation or high flow oxygen use, incidence of and mean duration of new oxygen use, incidence of and mean duration of new ventilator or ECMO use; number of non-invasive ventilation/high flow oxygen-free days, number of oxygenation-free days, number of ventilator/ECMO-free days); change from baseline in one or more clinical laboratory parameters (e.g., alanine transaminase (ALT), aspartate transaminase (AST), electrolytes, creatinine, glucose, hemoglobin, platelets, prothrombin time (PT), partial thromboplastin time (PTT), total bilirubin, and complete and white blood cell counts with differential; incidence of minor and major bleeding events; measures of arterial blood gas (ABG), measures of virology (viral load in nasopharyngeal swab test or Rutgers saliva test), measures of serology (antibody (IgM and IgG) production), measures of fecal load; and safety and tolerability as evidenced by AEs, vital signs (pulse, blood pressure, respiratory rate, body temperature), 12-lead ECG, Mobile Cardiac Outpatient Telemetry cardiac patch or hand-held ECG monitoring, continuous pulse oximetry, clinical laboratory tests (in addition to those listed above), physical examinations, and concomitant medications.

Example 3: Oral Dosage Forms. Tables 3 and 4 provide some example solid oral dosage forms that may be used to treat a viral infection according to any embodiment disclosed herein.

TABLE 3

| omponent | Amount Dose Unit (mg) | | | |
|---|---|---|---|---|
| | 1 | 2 | 3 | 4 |
| Dosage Type | High Dose, Slow Release | High Dose, Fast Release | Low Dose, Fast Release | Low Dose, Slow Release |
| Nafamostat mesylate | 100 | 100 | 1 | 1 |
| Hypromellose capsule | 1 unit | 1 unit | 1 unit | 1 unit |
| Microcrystalline cellulose spheres | 526.4 | 526.4 | 5.264 | 5.264 |
| Hypromellose | 100 | 100 | 1 | 1 |
| Amino methacrylate copolymer TYPE A (EUDRAGIT® RL) | 4.5 | 18.1 | 0.181 | 0.045 |
| Amino methacrylate copolymer TYPE A (EUDRAGIT® RS) | 86.2 | 72.6 | 0.726 | 0.862 |
| Triethyl citrate | 9.1 | 9.1 | 0.091 | 0.091 |
| Talc | 45.4 | 45.4 | 0.454 | 0.454 |
| Total | 871.6 | 871.6 | 8.72 | 8.72 |

TABLE 4

| Layer | Component | Amount Dose Unit (%) | | | |
|---|---|---|---|---|---|
| | | 5 | 6 | 7 | 8 |
| Core | Microcrystalline Cellulose Sphere | 63 | 60.5 | 60.5 | 60.5 |
| Active Agent Layer | Nafamostat mesylate | 12 | 11.5 | 11.5 | 11.5 |
| | Hydroxypropyl methyl cellulose | 12 | 11.5 | 11.5 | 11.5 |
| Controlled Release Layer | Amino methacrylate copolymer TYPE A (EUDRAGIT® RL) | 8 | 9 | 10.5 | 9.5 |
| | Amino methacrylate copolymer TYPE A (EUDRAGIT® RL) | 0 | 1.5 | 0 | 1 |
| | Triethyl citrate | 1 | 1 | 1 | 1 |
| | Talc | 4 | 5 | 5 | 5 |
| Total | | 100 | 871.6 | 8.72 | 8.72 |

1. A method of treating a viral infection in a subject comprising orally administering a therapeutically effective amount of nafamostat or a pharmaceutically acceptable salt thereof to the subject in need thereof, wherein the amount of nafamostat measurable in the plasma of the subject after 1 to 8 hours after administering is below about 5 ng/mL.

2. The method of claim 1, wherein the viral infection comprises infection by coronavirus, SARS-CoV, MERS-CoV, SARS-CoV-2, or another mutation of a coronavirus.

3. The method of claim 1, wherein the viral infection comprises infection by a coronavirus.

4. The method of claim 1, wherein the therapeutically effective amount of nafamostat is equivalent to the amount of nafamostat in about 100 mg to about 1500 mg of nafamostat mesylate.

5. The method of claim 4, wherein the effective amount of nafamostat or a pharmaceutically acceptable salt thereof is administered to the subject as one or more solid oral dosage forms comprising the nafamostat or a pharmaceutically acceptable salt thereof.

6. The method of claim 5, wherein the solid oral dosage form is a tablet, a bi-layer tablet, a capsule, a multiparticulate, a drug-coated sphere, a matrix tablet, or a multicore tablet.

7. The method of claim 5, wherein the solid oral dosage form comprises about 50 mg to about 200 mg of nafamostat mesylate or an equivalent amount of nafamostat or a pharmaceutically acceptable salt thereof.

8. (canceled)

9. (canceled)

10. The method of claim 1, wherein the administering comprises administering nafamostat in an amount equivalent to about 50 mg to about 500 mg nafamostat mesylate once, twice, or three times per day to the subject.

11. The method of claim 1, wherein the administering comprises administering nafamostat in an amount equivalent to at least about 0.1 mg nafamostat mesylate per kg of the subject’s weight per day.

12. The method of claim 1, wherein the administering comprises administering nafamostat in an amount equivalent to about 0.75 mg nafamostat mesylate per kg of the subject’s weight per day to about 20 mg per kg of the subject’s weight per day.

13. The method of claim 1, wherein the administering comprises administering nafamostat in an amount equivalent to about 1 mg nafamostat mesylate per kg of the subject’s weight per day to about 10 mg nafamostat mesylate per kg of the subject’s weight per day.

14. (canceled)

15. (canceled)

16. A method of inhibiting a viral infection in a subject, the method comprising orally administering a therapeutically effective amount of nafamostat or a pharmaceutically acceptable salt thereof.

17. The method of claim 12, wherein the administering occurs before a symptom of a viral infection is observed in the subject.

18. The method of claim 12, wherein the subject has been exposed to a virus.

19. The method of claim 12, wherein the viral infection comprises infection by a coronavirus, SARS-CoV, MERS-CoV, SARS-CoV-2, or another mutation of a coronavirus.

20. The method of claim 12, wherein the viral infection comprises infection by a coronavirus.

21. The method of claim 12, wherein the subject is aged 65 or older.

22. The method of claim 12, wherein the subject has one or more of cancer, chronic kidney disease, chronic obstructive pulmonary disease (COPD), down syndrome, heart failure, coronary artery disease, cardiomyopathy, sickle cell disease, type II diabetes mellitus, a BMI of 30 kg/m2 or higher, an immunocompromised condition from organ transplant, pregnancy, a smoking habit, inflammatory bowel disease, and irritable bowel syndrome.

23. The method of claim 12, wherein the subject has one or more of moderate-to-severe asthma, cerebrovascular disease, cystic fibrosis, hypertension, liver disease, dementia, Alzheimer’s Disease, pulmonary fibrosis, thalassemia, type I diabetes mellitus, a BMI of 25 kg/m2 to 30 kg/m2, and an immunocompromised condition from blood or bone marrow transplant, immune deficiency, HIV, or use of corticosteroid or other immune-weakening medicine.

24. The method of claim 12, wherein the administering comprising administering one or more solid oral dosage forms comprising nafamostat or a pharmaceutically acceptable salt of nafamostat.

25. The method of claim 12, wherein the solid oral dosage form is a tablet, a bi-layer tablet, a capsule, a multiparticulate, a drug-coated sphere, a matrix tablet, or a multicore tablet.

26. The method of claim 12, wherein the solid oral dosage form comprises about 50 mg to about 600 mg of nafamostat mesylate or an equivalent amount of nafamostat or a pharmaceutically acceptable salt thereof.

27. (canceled)

28. (canceled)

29. The method of claim 12, wherein the administering comprises administering nafamostat in an amount equivalent to about 200 mg to about 500 mg nafamostat mesylate once, twice, or three times per day to the subject.

30. The method of claim 12, wherein the administering comprises administering nafamostat in an amount equivalent to at least about 0.1 mg nafamostat mesylate per kg of the subject’s weight per day.

31. The method of claim 12, wherein the administering comprises administering nafamostat in an amount equivalent to about 0.75 mg nafamostat mesylate per kg of the subject’s weight per day to about 20 mg nafamostat mesylate per kg of the subject’s weight per day.

32. The method of claim 12, wherein the administering comprises administering nafamostat in an amount equivalent to about 1 mg per kg of the subject’s weight per day to about 10 mg per kg of the subject’s weight per day.

33. (canceled)

34. (canceled)

35. A method of reducing the risk of experiencing an adverse event associated with intravenous administration of nafamostat mesylate in a subject having a viral infection comprising orally administering a therapeutically effective amount of nafamostat mesylate to the subject, wherein the adverse event is one or more of allergic reaction, diabetic ketoacidosis, agranulocytosis, and hyperkalemia.

36. (canceled)

37. (canceled)
